(19) United States
(12) Patent Application Publication
(10) Pub. No.: US 2009/0054455 A1
(43) Pub. Date: Feb. 26, 2009

(54) ARIPIPRAZOLE CO-CRYSTALS

(75) Inventors: Surya Narayana Devarakonda, Hyderabad (IN); Krishnamurthi Vyas, Hyderabad (IN); Sivakumar Reddy Bommareddy, Hyderabad (IN); Pratap Reddy Padi, Hyderabad (IN); Balaji Raghupathy, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500 016, A.P. (IN); DR. REDDY'S LABORATORIES, INC., Bridgewater, NJ (US)

(21) Appl. No.: 12/278,022

(22) PCT Filed: Feb. 2, 2007
(86) PCT No.: PCT/US07/61552

§ 371 Date: Aug. 1, 2008

Related U.S. Application Data
(60) Provisional application No. 60/803,085, filed on May 24, 2006.

(30) Foreign Application Priority Data
Feb. 3, 2006 (IN) 173/CHE/2006

(51) Int. Cl. A61K 31/497

(52) U.S. Cl. 514/253.08

(57) ABSTRACT

Co-crystals comprising aripiprazole and fumaric acid.

### INTRODUCTION TO THE INVENTION

The present invention relates to co-crystals comprising aripiprazole and fumaric acid and processes for co-crystal preparation.

Aripiprazole is the adopted name for the compound having a chemical name 7-[4-[4-(2,3-dichlorophenyl)-1-piperazinyl]butoxy]-3,4-dihydrocarbostyril, structurally represented by Formula I.

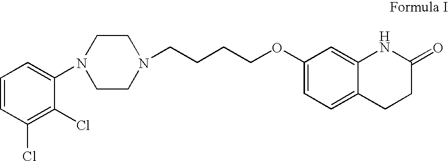

Aripiprazole is a psychotropic drug useful for the treatment of schizophrenia and is the sixth, and most recent, of the second generation antipsychotic medications. It is available in the market under the brand name ABILIFY® in the form of tablets of 5 mg, 10 mg, 15 mg, 20 mg, and 30 mg strengths.

U.S. Pat. Nos. 4,734,416 and 5,006,528 disclose aripiprazole and its pharmaceutically acceptable salts, processes to make them and compositions containing them. The patents also give a process for the preparation of an aripiprazole fumarate salt by reacting approximately equimolar amounts of aripiprazole and fumaric acid.

Various other crystalline forms, hydrates and solvates of aripiprazole have been described in International Application Publication Nos. WO 2006/079548 A1, WO 2006/077584 A2, WO 2006/053781 A1, WO 2006/053780 A1, WO 2006/030446 A1, WO 2006/012237 A2, WO 2005/009990 A1, WO 2005/058835 A2, WO 2004/106322 A2, WO 2004/083183 A1, and WO 2003/026659 A1.

The literature has not described a co-crystal of aripiprazole. Co-crystals are combinations of two or more distinct molecules arranged to create a unique crystal form.

Co-crystals include two or more different components and often rely on hydrogen bonded assemblies between neutral molecules. Co-crystals with the same active pharmaceutical ingredient will have strikingly different pharmaceutical properties like melting point, solubility, dissolution, moisture uptake, chemical stability etc. depending on the nature of the second component.

The co-crystal complexes of a number of drugs exhibit different dissolution characteristics, generally enhanced dissolution, resulting frequently in an enhanced bioavailability profile, compared to any polymorph of the same drug.

Aripiprazole presents certain challenges for formulation as a rapid-onset dosage form, particularly as a rapid-onset oral dosage form. For example, aripiprazole has a very low solubility in aqueous media (being practically insoluble) and therefore is not readily dissolved and dispersed for rapid absorption in the gastrointestinal tract when administered orally, for example in tablet form.

The bioavailability of an orally administered drug, as measured by its entry into systemic circulation in the bloodstream, depends on at least two fundamental processes: drug dissolution in gastrointestinal fluids (in vivo drug release) and subsequent systemic absorption of the dissolved drug. Several factors influence dissolution of a drug from its carrier, including surface area of the drug presented to the dissolution solvent medium, solubility of the drug substance in the solvent medium, and driving forces of the saturation concentration of dissolved materials in the solvent medium.

Towards this end, it has been the endeavor of pharmaceutical scientists to provide new forms of aripiprazole, more specifically, a thermodynamically stable form which would have the strengths of the crystalline forms, viz. thermodynamic stability, and those of the amorphous form, viz. enhanced solubility, rapid onset of action and an enhanced bioavailability.

Consequently, there is a need for soluble forms of aripiprazole that can be readily formulated for use in various modes of administration, including parenteral and oral administration. Co-crystal complexes of aripiprazole would add a powerful tool in the treatment of central nervous system disorders.

The present invention provides co-crystals of aripiprazole and fumaric acid which are stable and are reproducible on an industrial scale.

### SUMMARY OF THE INVENTION

The present invention relates to co-crystals comprising aripiprazole and fumaric acid and processes for its preparation.

One aspect of the present invention provides a co-crystal comprising aripiprazole and fumaric acid. A co-crystal can be characterized by its X-ray powder diffraction (“XRPD”) patterns, differential scanning calorimetry (“DSC”) curves, infrared (“IR”) absorption spectra, and ′H NMR spectra.

Another aspect of the present invention provides a process for the preparation of a co-crystal comprising aripiprazole and fumaric acid. In an embodiment, a process for the preparation of a co-crystal comprising aripiprazole and fumaric acid comprises:

a) providing a solution of aripiprazole and fumaric acid in a suitable solvent;

b) adding an anti-solvent to the solution obtained in step a);

c) recovering a co-crystal comprising aripiprazole and fumaric acid.

Still another aspect of the present invention provides a pharmaceutical composition comprising co-crystals of aripiprazole and fumaric acid and their combination with one or more pharmaceutically acceptable excipients.

In an aspect, a co-crystal has a molar ratio of fumaric acid to aripiprazole about 0.5.

### BRIEF DESCRIPTION OF THE DRAWINGS

FIG. 1 is an X-ray powder diffraction pattern of co-crystals of aripiprazole and fumaric acid prepared in Example 1.

FIG. 2 is a differential scanning calorimetric curve of co-crystals of aripiprazole and fumaric acid prepared in Example 1.

FIG. 3 is an infrared absorption spectrum of co-crystals of aripiprazole and fumaric acid prepared in Example 1.

FIG. 4 is a ¹H NMR spectrum of co-crystals of aripiprazole and fumaric acid prepared in Example 1.

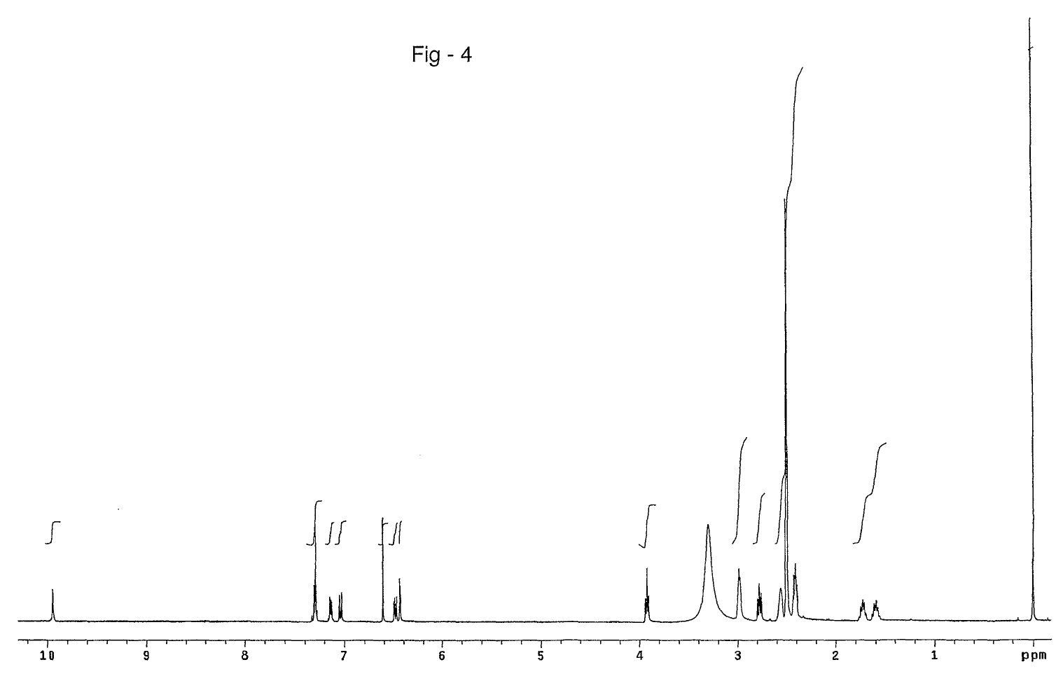

### DETAILED DESCRIPTION OF THE INVENTION

The present invention relates to co-crystals comprising aripiprazole and fumaric acid and processes for co-crystal preparation.

One aspect of the present invention provides co-crystals of aripiprazole and fumaric acid characterized by their X-ray powder diffraction (“XRPD”) patterns, differential scanning calorimetry (“DSC”) curves, infrared (“IR”) absorption spectra, and ′H NMR spectra.

The molar ratio of fumaric acid in the co-crystals of aripiprazole and fumaric acid may range from about 0.5 to about 10 moles, per mole of aripiprazole.

In one embodiment, the molar ratio of fumaric acid in the co-crystals comprising aripiprazole and fumaric acid is about 0.5 moles, per mole of aripiprazole.

The XRPD data reported herein were obtained using Cu Kα-1 radiation, having the wavelength 1.541 Å, and were generated using a Bruker Axe, D8 Advance Powder X-ray Diffractometer.

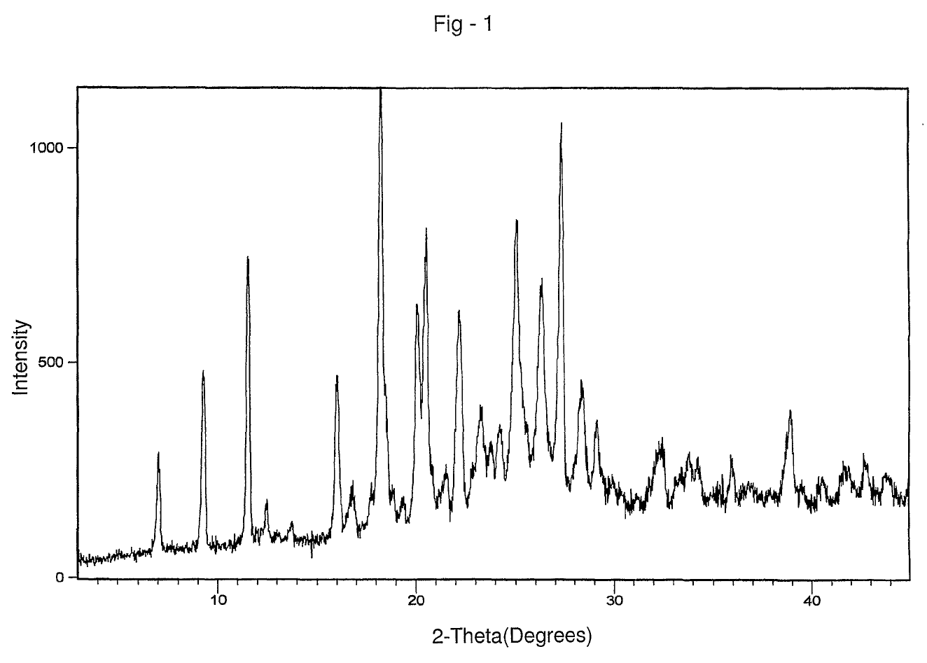

Co-crystals of aripiprazole and fumaric acid are characterized by an XRPD pattern substantially in accordance with the pattern of FIG. 1. Co-crystals of aripiprazole and fumaric acid are also characterized by an XRPD pattern having significant peaks at about 9.3, 11.6, 18.2, 20.0, 20.5, 22.1, 23.2, 25.0, and 38.9, ±0.2 degrees 2θ. The pattern is also characterized by the additional XRPD peaks at about 26.2, 29.1, and 16.0, ±0.2 degrees 2θ.

The infrared (IR) spectra of co-crystals of aripiprazole and fumaric acid have been recorded on a Perkin Elmer System Spectrum 1 model spectrophotometer, between 450 cm⁻¹ and 4000 cm⁻¹, with a resolution of 4 cm⁻¹ in a potassium bromide pellet, the test compound being at the concentration of 1% by mass.

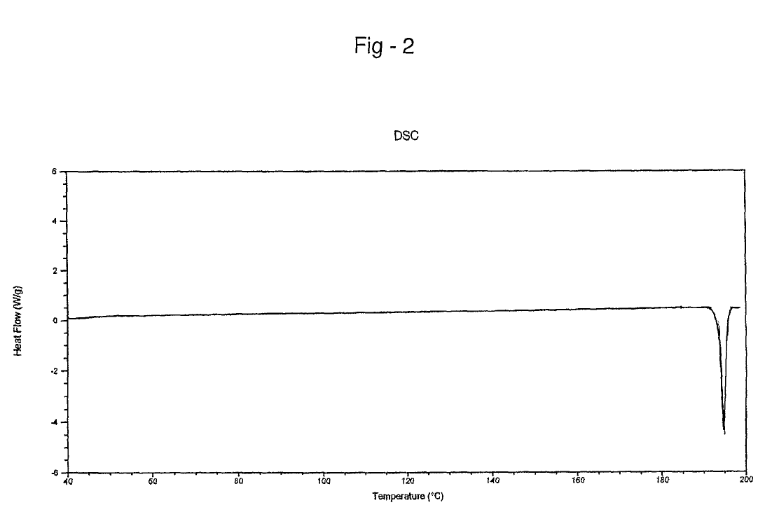

Co-crystals of aripiprazole and fumaric acid are characterized by an infrared absorption spectrum in potassium bromide comprising peaks at about 636, 783, 855, 956, 1170, 1198, 1378,1449, 1628, 1688, 2943, and 3197, ±5 cm⁻¹. Co-crystals of aripiprazole and fumaric acid are also characterized by its infrared absorption spectrum in potassium bromide substantially in accordance with the spectrum of FIG. 2.

Differential scanning calorimetric analysis was carried out in a DSC Q1000 model from TA Instruments with a ramp of 5° C./minute. The starting temperature was 40° C. and ending temperature was 200° C.

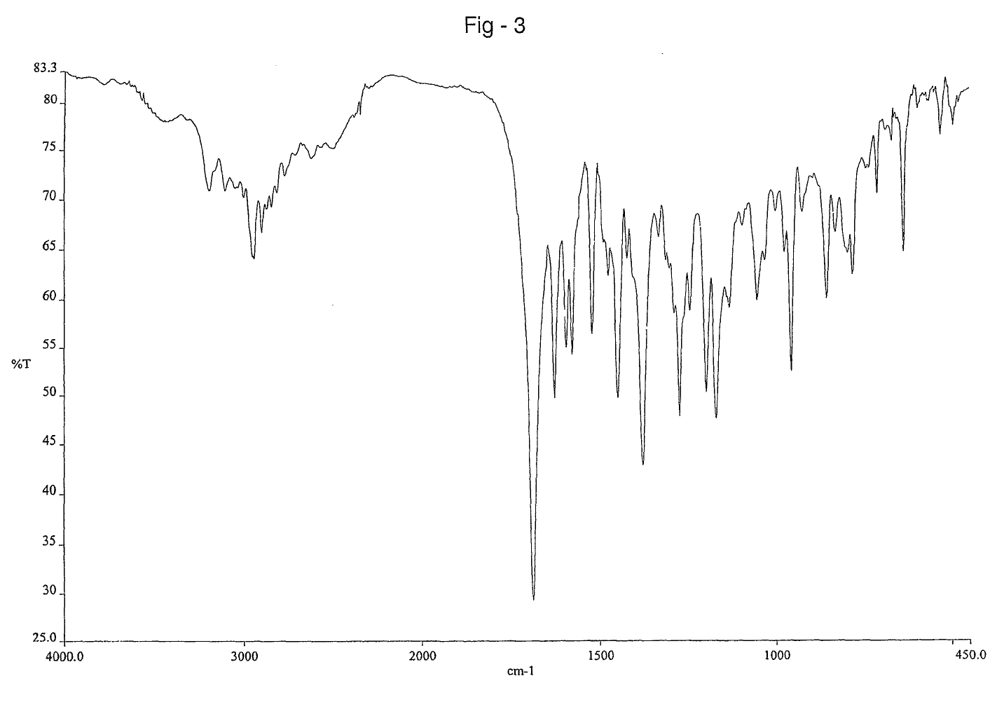

Co-crystals of aripiprazole and fumaric acid have a characteristic differential scanning calorimetry curve substantially in accordance with FIG. 3, having an endotherm at about 194° C.

Another aspect of the present invention provides a process for the preparation of co-crystals comprising aripiprazole and fumaric acid.

The co-crystals can be constructed through several modes of molecular recognition including hydrogen-bonding, π (pi)-stacking, guest-host complexation and Van Der Waals interactions. Of the interactions listed above, hydrogen-bonding is the dominant interaction in the formation of the co-crystal, whereby a non-covalent bond is formed between a hydrogen bond donor of one of the moieties and a hydrogen bond acceptor of the other. Co-crystals of the present invention are considered to be those where hydrogen bonding occurs between the co-crystal forming agent and active pharmaceutical ingredient.

Fumaric acid is the co-crystal forming agent in the present invention. Other acids like nicotinic acid, benzoic acid, succinic acid, and L-tartaric acid have not formed co-crystals with aripiprazole using the process of the present invention.

In the process according to the invention, there is a need to contact the active pharmaceutical ingredient aripiprazole with the co-crystal forming compound fumaric acid. This may involve either solubilizing aripiprazole and adding fumaric acid, or solubilizing fumaric acid and adding aripiprazole to it.

In an embodiment, a process for the preparation of co-crystals of aripiprazole and fumaric acid comprises:

a) providing a solution of aripiprazole and fumaric acid in a suitable solvent;

b) adding an anti-solvent to the solution obtained in step a);

c) recovering co-crystals comprising aripiprazole and fumaric acid.

Step a) involves providing a solution of aripiprazole and fumaric acid in a suitable solvent.

A solution of aripiprazole and fumaric acid may be obtained by dissolving aripiprazole and fumaric acid in a suitable solvent, or such a solution may be obtained by adding fumaric acid to a reaction mixture in which aripiprazole was formed

When the solution is prepared by dissolving aripiprazole and fumaric acid in a solvent, any form of aripiprazole such as any crystalline form of aripiprazole including any solvates and hydrates may be utilized for preparing the solution.

When the solution of aripiprazole along with fumaric acid is prepared, the order of charging the two ingredients into the solvent is not critical for the product obtained. A specific order may be preferred with respect to the equipment actually used and will be easily determined by a person skilled in the art. In any case, the aripiprazole must be completely soluble in the solvent of the invention and should provide a clear solution.

Suitably, aripiprazole is added first and, after obtaining a clear solution of aripiprazole in the solvent, fumaric acid is added at the same temperature used for dissolution, or it can be cooled to lower temperatures than the temperatures used for the dissolution of aripiprazole.

The mole ratio of fumaric acid used to that of aripiprazole may range from about 0.5 to about 10 moles per mole of aripiprazole.

Fumaric acid can be added directly, or it can be dissolved in a solvent before addition to the solution of aripiprazole.

Suitable solvents which can be used for preparing the solution include but are not limited to: aprotic polar solvents such as N,N-dimethylformamide (DMF), dimethylsulfoxide (DMSO), N,N-dimethylacetamide, acetonitrile and the like; ethers such as tetrahydrofuran, 1,4-dioxane and the like; alcoholic solvents such as methanol, ethanol, isopropanol, n-propanol, n-butanol, tertiary-butyl alcohol, ethylene glycol, and the like; and mixtures thereof.

The dissolution temperatures can range from about 20 to 120° C. depending on the solvent used for dissolution. Any other temperature is also acceptable as long as a clear solution is obtained and the ariprazole stability is not compromised.

The quantity of solvent used for dissolution depends on the solvent and the dissolution temperature adopted. The concentration of aripiprazole in the solution may generally range from about 0.1 to about 10 g/ml in the solvent.

These lists of solvents are merely representative of those that can be used, and the lists are not intended to be exhaustive.

The solution may optionally be treated with materials such as carbon or sodium sulfate for clarification.

Optionally, the solution obtained above can be filtered to remove any undissolved particles followed by further processing. The undissolved particles can be removed suitably by filtration, centrifugation, decantation, and other techniques. The solution can be filtered by passing through paper, glass fiber, or other membrane material, or a bed of a clarifying agent such as celite. Depending upon the equipment used and the concentration and temperature of the solution, the filtration apparatus may need to be preheated to avoid premature crystallization.

Step b) involves adding an anti-solvent to the solution obtained in step a).

Suitable anti-solvents include, but are not limited to: nitriles such as acetonitrile, propionitrile and the like; ethers such as diethyl ether, dimethyl ether, di-isopropyl ether, methyl tertiary-butyl ether, tetrahydrofuran, 1,4-dioxane and the like; hydrocarbons such as toluene, xylene, n-heptane, cyclohexane, n-hexane and the like; and mixtures thereof.

Suitably, the anti-solvent should have less polarity than the solvent used for dissolution of aripiprazole and fumaric acid in step a).

The anti-solvent can be added to the solution obtained in step b) at any temperature ranging from about 20° C. to about 60° C. The anti-solvent helps in initiating the isolation of the desired product.

For isolation to occur, the reaction mass may be maintained further at temperatures lower than the dissolution temperatures such as for example below about 10° C. to about 25° C., for a period of time as required for a more complete isolation of the product. The exact cooling temperature and time required for complete crystallization can be readily determined by a person skilled in the art and will also depend on parameters such as concentration and temperature of the solution or slurry.

Optionally isolation may be initiated or enhanced by methods such as cooling, seeding, partial removal of the solvent from the solution, or a combination thereof.

Step c) involves recovering the co-crystals comprising aripiprazole and fumaric acid.

The method by which the solid material is recovered from the final mixture, with or without cooling below the operating temperature, can be any of techniques such as filtration by gravity or by suction, decantation, centrifugation, and the like. The crystals so isolated will usually carry a small proportion of occluded mother liquor containing a higher percentage of impurities. If desired the crystals can be washed with a solvent to wash out the mother liquor.

Optionally, the obtained product is further dried. Drying can be carried out at reduced pressures, such as below about 200 mm Hg or below about 50 mm Hg, at temperatures such as about 35° C. to about 70° C., depending on the volatility of the solvent and antisolvent used. Drying may be carried out for shorter or longer periods of time depending on the desired product to be obtained.

Drying can be suitably carried out in a tray dryer, vacuum oven, air oven, or using a fluidized bed drier, spin flash dryer, flash dryer and the like.

The process of the present invention provides stable co-crystals of aripiprazole with fumaric acid. The term “stable crystalline form” refers to stability of the crystalline form under the standard temperature and humidity conditions of testing of pharmaceutical products, wherein the stability is indicated by preservation of the original polymorphic form.

Co-crystals of aripiprazole with fumaric acid obtained in this invention contain less than about 5000 ppm, or less than about 3000 ppm, or less than about 1000 ppm of methanol, and less than about 200 ppm, or less than about 100 ppm of other individual residual organic solvents.

Still another aspect of the present invention provides a pharmaceutical composition comprising co-crystals of aripiprazole and fumaric acid, together with one or more pharmaceutically acceptable excipients.

The pharmaceutical composition comprising co-crystals of aripiprazole and fumaric acid of the present invention may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of present invention aripiprazole is a useful active ingredient in the range of 5 mg to 10 mg, or 15 mg to 20 mg, or 30 mg per dosage unit.

Certain specific aspects and embodiments of this invention are described in further detail by the examples below, which examples are provided only for the purpose of illustration and are not intended to limit the scope of the appended claims in any manner.

### EXAMPLE 1

### Preparation of Co-Crystals of Aripiprazole and Fumaric Acid

1 g of aripiprazole anhydride prepared according to Example 2 of US 2005/0277650 and 100 ml dimethylformamide (DMF) were taken into a clean and dry round bottom flask followed by stirring at 25° C. for 10 minutes. 1 g of fumaric acid was added followed by stirring for 15 minutes. To the resultant solution 700 ml of acetonitrile was added slowly to cause precipitation of compound. The resultant suspension was stirred at 25° C. for 60 minutes followed by filtration of separated solid. The solid obtained was dried under a vacuum of about 1 torr at 70° C. for 6 hours to afford 0.6 g of the title compound.

¹H NMR spectra of a sample confirmed the presence of 1 molecule of fumaric acid, per 2 molecules of aripiprazole.

### EXAMPLE 2

### Preparation of Co-Crystals of Aripiprazole and Fumaric Acid

0.5 g of aripiprazole anhydride prepared according to Example 2 of US 2005/0277650 and 22 ml of dimethylsulfoxide (DMSO) were taken into a clean and dry round bottom flask followed by stirring for 15 minutes. After dissolution of the solid, 0.5 g of fumaric acid was added and the mass was stirred at 25° C. for 15 minutes. To the resultant solution 80 ml of acetonitrile was added slowly at 25° C. The resultant suspension was stirred at 25° C. for 15 minutes followed by filtration of the separated solid. The solid was dried at 70° C. under a vacuum of about 1 torr for 5 hours to yield 0.2 g of the title compound.

¹H NMR spectra of a sample confirmed the presence of 1 molecule of fumaric acid, per 2 molecules of aripiprazole.

### EXAMPLE 3

### Preparation of Co-Crystals of Aripiprazole and Fumaric Acid

45 g of aripiprazole (0.1 mole) and 550 ml of dimethylformamide were taken into a round bottom flask and stirred at 30° C. for 30 minutes. 6 g of fumaric acid (0.052 moles) was dissolved in 15 ml of dimethylformamide and stirred for 5 minutes. The solution of fumaric acid in dimethylformamide was added to the solution of aripiprazole prepared above at 30° C. and stirred for 15 minutes. 200 ml of acetonitrile was added to the above reaction mass at 30° C. and stirred for 20 minutes. The reaction mass was then cooled to 5° C. and maintained for 30 minutes. The separated solid was filtered and the wet compound was dried at 62° C. under a vacuum of 650 mm Hg for 12 hours to get 40.4 kg of co-crystals having 0.5 moles of fumaric acid per mole of aripiprazole.

### EXAMPLE 4

### Stability of Co-Crystals of Aripiprazole and Fumaric Acid

Aripiprazole was prepared according to the process given in Example 2 and was packaged in a self-sealing polyethylene bag. The material was stored for 3 months under normal atmospheric conditions at room temperature and checked for polymorphic stability.

The material was found to retain its polymorphic form after three months of holding, as indicated by maintenance of the original XRPD pattern.

1-12. (canceled)

13. A composition prepared by a process comprising providing a solution of aripiprazole and fumaric acid in a solvent, and adding an anti-solvent to the solution.

14. The composition of claim 13, wherein a solvent comprises an aprotic polar solvent, an ether, an alcohol, or a mixture of any two or more thereof.

15. The composition of claim 13, wherein a solvent comprises N,N-dimethylformamide.

16. The composition of claim 13, wherein a solvent comprises dimethylsulfoxide.

17. The composition of claim 13, wherein an antisolvent comprises a nitrile, an ether, a hydrocarbon, or a mixture of any two or more thereof.

18. The composition of claim 13, wherein a solvent comprises an aprotic polar solvent and an antisolvent comprises a nitrile.

19. The composition of claim 13, wherein a molar ratio of fumaric acid to aripiprazole is about 0.5.

20. The composition of claim 13, wherein an antisolvent comprises a nitrile.

21. The composition of claim 13, wherein an antisolvent comprises acetonitrile.

22. The composition of claim 13, having an X-ray powder diffraction pattern substantially in accordance with the pattern of FIG. 1.

23. A solid pharmaceutical dosage form, comprising the composition of claim 13 and at least one pharmaceutically acceptable excipient.

24. The pharmaceutical formulation of claim 13, wherein a molar ratio of fumaric acid to aripiprazole is about 0.5.

25. A composition prepared by a process comprising providing a solution of aripiprazole and fumaric acid in a solvent comprising an aprotic polar solvent, an ether, an alcohol, or a mixture of any two or more thereof, and adding a nitrile to the solution.

26. The composition of claim 25, wherein a solvent comprises N,N-dimethylformamide.

27. The composition of claim 25, wherein a solvent comprises dimethylsulfoxide.

28. The composition of claim 25, wherein a nitrile comprises acetonitrile.

29. The composition of claim 25, wherein a molar ratio of fumaric acid to aripiprazole is about 0.5.

30. The composition of claim 25, having an X-ray powder diffraction pattern substantially in accordance with the pattern of FIG. 1.

31. A composition prepared by a process comprising providing a solution of aripiprazole and fumaric acid in a solvent comprising N,N-dimethylformamide or dimethylsulfoxide, and adding a nitrile to the solution.

32. The composition of claim 31, wherein a nitrile comprises acetonitrile.

33. The composition of claim 31, wherein a molar ratio of fumaric acid to aripiprazole is about 0.5.

34. The composition of claim 31, having an X-ray powder diffraction pattern substantially in accordance with the pattern of FIG. 1.
